(19) United States
(12) Patent Application Publication
(10) Pub. No.: US 2005/0221435 A1
(43) Pub. Date: Oct. 6, 2005

(54) Biological material for preparing pharmaceutical compositions intended for treating mammals

(76) Inventors: Bruce Acres, Strasbourg (FR); Stephane Paul, Strasbourg (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 11/124,097

(22) Filed: May 9, 2005

Related U.S. Application Data
(63) Continuation of application No. 09/806,721, filed on Jul. 11, 2001.

(30) Foreign Application Priority Data
Oct. 22, 1998 (FR) 98/13279

(51) Int. Cl. C12P 21/06; C07H 21/04; C12N 5/06; C12N 15/867; C12N 15/86

(52) U.S. Cl. 435/69.1; 435/456; 435/326; 536/23.53; 530/387.1

(57) ABSTRACT

The invention concerns a biological material for preparing pharmaceutical compositions for treating mammals including either a nucleic acid sequence, containing at least a gene of therapeutic interest and elements expressing the gene in vivo in target cells genetically modified by one such nucleic sequence, or at least a target cell not producing antibodies naturally, genetically modified in vitro by at least one such nucleic acid sequence. The gene of therapeutic interest codes for all or part of an antibody expressed at the surface of the target cell, and such an antibody is capable of fixing itself to a polypeptide present at the surface of a cytotoxic effector cell or a T lymphocyte helper involved in the process activating such a cell.

The present invention relates to the field of gene therapy applied to specific immunotherapy, more particularly in the context of treatments for diseases in which the agent responsible is a pathogenic organism, such as in particular a bacterial, parasitic or viral agent, or in the context of cancer treatment. More particularly, the invention relates to the transfer, into tumor cells or cells infected with a pathogenic agent, of nucleic acid sequences encoding all or part of antibodies, such that the cells genetically modified with these nucleic acid sequences express said antibodies at their surface, and more particularly to antibodies capable of binding to a polypeptide which is present at the surface of a cytotoxic effector cell or of a helper T lymphocyte, and which is involved in the process of activation of such a cell.

For a long time, gene therapy has been suggested for correcting the conditions observed in the context of genetic diseases. These diseases are explained in particular by a dysfunction of the expression of specific genes or by the expression of nonfunctional mutated polypeptides in at least one cell type. Gene therapy consists, in this case, in transferring into specific target cells which are extracted from and then reintroduced into the human body, or directly into the affected organs, the genetic information capable of correcting the observed defect. It may be, for example, the gene encoding the CFTR protein, in the case of cystic fibrosis, or the gene encoding dystrophin in the case of Duchenne myopathy. In the context of this approach, the genetic information is introduced either in vitro into a cell which has been extracted from the organ, the modified cell then being reintroduced into the body (ex vivo method), or in vivo directly into the appropriate tissue. Many publications describe the use of gene therapy protocols in order to obtain, in the target cells, the expression of a protein having a therapeutic value by introducing the corresponding genetic information.

However, the value of this type of therapy is not limited to the treatment of purely genetic disorders, and can also allow the elimination of tumors, or of pathogenic agents, such as bacterial or viral agents, or of cells infected with such pathogenic agents, or failing that, to slow down their progression.

The immune response directed against a specific antigen can be divided into two distinct categories, one making use of antibodies (humoral-type immune response), the other making use of cytotoxic effector cells such as, for example, macrophages, cytotoxic lymphocytes (CTLs) or killer cells (NK), and helper T lymphocytes, in particular LTCD4s (cellular-type immune response). More particularly, the two types of response differ in that the antibodies recognize the antigens in their three-dimensional form, whereas the T lymphocytes, for example, recognize peptide portions of said antigens, combined with glycoproteins encoded by the genes of the major histocompatibility complex (or MHC), in particular the genes of the type I major histocompatibility complex which are expressed ubiquitously at the surface of cells, or the genes of the type II major histocompatibility complex which are expressed specifically at the surface of the cells involved in antigen presentation (APCs).

According to a first aspect, the cellular-type immune response is characterized in that T cells of CD4+ type (“helper” T cells), subsequent to a well known activation phenomenon (for a review see Alberola-Ila, 1997, Annu. Rev. Immunol., 15, 125-154), produce cytokines which in turn induce the proliferation of APC cells capable of producing said cytokines; the cellular differentiation of B lymphocytes capable of producing specific antibodies; and the stimulation of cytotoxic T lymphocytes (CTLs). According to a second aspect of the cellular immune response, the cytotoxic effector cells, such as for example lymphocytes of CD8+ type (CTLs) are activated a) after interaction with antigenic peptides bound to and presented by the glycoproteins borne by ubiquitous cells and encoded by the genes belonging to the MHC I system, and b) possibly by the cytokines produced by the CD4+ s. The CTLs thus activated are then capable of destroying the cells expressing said antigenic peptide.

In the particular case of cancers, Hellstrom et al., (1969, Adv. Cancer Res. 12, 167-223) have shown that the body's defense with regard to tumors is based most particularly on the immune response using T lymphocytes, in particular cytotoxic T lymphocytes. However, many studies have shown that most of these immune effectors, which may or may not be specific, are ineffective in allowing the elimination or arrest of progression of a tumor. It is for this reason that it is desirable to have a method for stimulating the immune response directed against tumors, and more particularly the response involving cytotoxic lymphocytes CTLs, in order to have more effective methods for preventing or for treating cancerous conditions. In an identical manner, it has been shown that the immune system is often inefficient in the case of infections, in particular viral infections; see for example the case of infections due to HIV (Human Immunodeficiency Virus).

According to a first alternative, it has been proposed to adapt the methods of gene therapy already well known and to transfer into the target cells, more particularly the cancerous cells, immunostimulatory genes (immunotherapy) which are capable of inducing or of activating a cell-mediated immune response with regard to the tumor, of genes encoding cytokines (Colombo et al., 1994, Immunology Today, 15, 48-51), of cytotoxic genes conferring toxicity on the cells expressing them, for example the tk gene of the type 1 Herpes Simplex virus (HSV-1), or of anti-oncogenes, such as for example the gene associated with retino-blastoma or p53, or of polynucleotides capable of inhibiting the activity of an oncogene, such as for example antisense molecules, or ribozymes capable of degrading messenger RNAs specific for oncogenes. However, in the majority of cases, the cells thus modified lack specificity with respect to the tumor, and do not allow a satisfactory therapeutic approach.

Another approach has also been proposed which allies the advantages of gene therapy and the use of specific antibodies. During the last few years, many tumor antigens have been characterized which have more particularly enabled the identification of antibodies, in particular monoclonal antibodies, specific for these antigens. Moreover, the in vitro production of antibodies, of antibody fragments or of antibody derivatives such as chimeric antibodies, by genetic engineering, in eukaryotic cells, has been described (EP 120 694 or EP 125 023). Thus, many therapeutic strategies have been proposed, for example for treating or preventing B lymphomas (Yefenof et al., 1993, Current Opinion in Immunology, 5, 740-744), which are based on the administration to the patient of therapeutic antibodies targeting tumor antigens in order to neutralize the agent causing the disease. Unfortunately, these antibodies, although very useful for detecting and diagnosing cancers, have proved to be unsatisfactory from a therapeutic point of view, since they lead, for example, to limited immune reactions which are directed only against immunodominant epitopes or against antigens with a high variability.

International patent application (WO 94/29446) describes the intracellular expression of DNA sequences encoding antibodies. This approach makes it possible to envisage gene therapy which is based on targeting cellular components which are not accessible via vaccination methods, and is characterized in that the approach described does not involve the development of an immune response, acts intracellularly and, consequently, does not allow the effective treatment of tumors.

International patent application (WO 98/31808) on the contrary relates to the expression, in vivo, of genes encoding antibodies or antibody fragments by cells capable of secreting said antibodies into the blood circulation of mammals carrying the cells which are genetically modified with the gene encoding the antibody. The advantage of this invention is based on the possibility of maintaining a basal antibody level in the patient treated, over the long term.

We have now shown that it is possible to redirect the cellular immune response by expressing at the surface of target cells, in particular tumor cells or cells infected with a pathogenic agent, all or part of an antibody capable of binding to a polypeptide which is present at the surface of cytotoxic effector cells or of helper T lymphocytes. More particularly, we have shown that, according to the present invention, these genetically modified target cells make it possible to direct the activation of cytotoxic effector cells or of helper T lymphocytes, to increase the cytotoxic effect of these cells, which are in particular activated, with regard to the target cells, and to stimulate the cell-mediated immune response which is produced naturally with regard to genetically modified or unmodified tumor or infected cells. This can thus cause the lysis and elimination of said target cells and of neighboring cells and, in the end, the elimination of the tumor or of the infection.

The present invention relates, firstly, to a biological material for preparing pharmaceutical compositions intended for treating mammals, comprising:
either at least one nucleic acid sequence containing at least one gene of therapeutic interest and elements which ensure the expression of said gene in vivo in target cells intended to be genetically modified with said nucleic acid sequence;
or at least one mammalian cell which does not naturally produce antibodies and which is genetically modified in vitro with at least one nucleic acid sequence above,
   characterized in that said gene of therapeutic interest encodes all or part of an antibody which will be expressed at the surface of said mammalian cell, and in that said antibody is capable of binding to a polypeptide which is present at the surface of a cytotoxic effector cell or of a helper T lymphocyte, and which is involved in the process of activation of such a cell.

The expression “nucleic acid sequence” is intended to refer to a synthetic or natural isolated, linear or circular, double- or single-stranded DNA and/or RNA fragment which designates a specific series of modified or unmodified nucleotides, making it possible to define a fragment or region of a nucleic acid without any limitation in size. According to one preferred embodiment, it is a nucleic acid chosen from the group consisting of a cDNA; a genomic DNA; a plasmid DNA and a messenger RNA.

According to the invention, said “nucleic acid sequence” comprises at least one “gene of therapeutic interest” and elements for the expression of said gene of interest. Such a “gene of therapeutic interest” encodes in particular all or part of a native transmembrane antibody, or a derivative of such an antibody, provided that said antibody, antibody derivative or fragment is expressed at the surface of the genetically modified mammalian target cell, and that said antibody is capable of binding to a polypeptide which is present at the surface of a cytotoxic effector cell or of a helper T lymphocyte, and which is involved in the process of activation of such a cell. More particularly, the term antibody “fragment” is intended to refer to F(ab)₂, Fab′, Fab, sFv fragments (Blazar et al., 1997, Journal of Immunology, 159, 5821-5833; Bird et al., 1988, Science, 242, 423-426) of a native antibody, and the term “derivative” is intended to mean, for example, a chimeric derivative of such an antibody (see for example the chimeras the anti-CD3 mouse/human antibodies in Arakawa et al., 1996, J. Biochem., 120, 657-662 or the immunotoxins such as sFv-toxin of Chaudary et al., 1989, Nature 339, 394-397). The term “transmembrane antibody” is intended to refer to an antibody in which at least the functional region which is capable of recognizing and of binding to its specific antigen is expressed at the surface of the target cells so as to allow said recognition and binding. More particularly, the antibodies according to the present invention consist of fusion polypeptides comprising the amino acids which define said functional region and an amino acid sequence (transmembrane polypeptide) which allows the anchoring within the double membrane lipid layer of the target cell, or at the external surface of this bilayer. The nucleic acid sequences encoding many transmembrane polypeptides are described in the literature. According to one preferred case of the invention, said transmembrane polypeptide is selected from the group consisting of a glycoprotein, a lipoprotein and of a receptor such as all or part of the complexes mentioned later in the application (CD4, Fc, HIV gp160, for example), and more particularly from the group consisting of the rabies virus glycoprotein (French patent application No. FR 97 09152), CD4 (Weijtens et al., 1998, Gene Therapy, 5, 1195-1203), gp160 and Fc. According to one absolutely advantageous case, the nucleic acid sequence encoding the antibody heavy chain will be fused with the nucleic acid sequence encoding a said transmembrane polypeptide.

The expression “elements which ensure the expression of said gene in vivo” is intended to refer to the elements required in order to ensure the expression of said gene after its transfer into a target cell. They are in particular promoter sequences and/or regulatory sequences which are effective in said cell and, optionally, the sequences required to allow the expression, at the surface of the target cells, of said polypeptide. The promoter used can be a viral, ubiquitous or tissue-specific promoter, or alternatively a synthetic promoter. By way of example, mention will be made of promoters such as the promoters of the RSV (Rous Sarcoma Virus), MPSV, SV40 (Simian Virus) or CMV (cytomegalovirus) viruses, or of the vaccinia virus, and the promoters of the gene encoding muscle creatine kinase, encoding actin, and encoding lung surfactant. It is also possible to choose a promoter sequence which is specific for a given cell type, or which can be activated under defined conditions. The literature provides a large amount of information relating to such promoter sequences. Moreover, said nucleic acid can comprise at least two sequences, which may be identical or different, having transcriptional promoter activity and/or at least two genes, which may be identical or different, located, with respect to each other, contiguously, far apart, in the same orientation or in reverse orientation, provided that the transcriptional promoter function or the transcription of said genes is not affected. Similarly, in this type of nucleic acid construct, it is possible to introduce “neutral” nucleic acid sequences or introns, which do not adversely affect transcription and are spliced out before the translation step. Such sequences and their uses are described in the literature (WO 94/29471). Said nucleic acid may also contain sequences required for intracellular transport, for replication and/or for integration, for transcription or translation. Such sequences are well known to persons skilled in the art. Moreover, the nucleic acids which can be used according to the present invention may also be nucleic acids which are modified in such a way that it is not possible for them to integrate into the genome of the target cell, or nucleic acids stabilized using agents, such as for example spermine, which, as such, do not have any effect on transfection efficiency.

According to one embodiment of the invention, the nucleic acid sequence is a naked DNA or RNA sequence, i.e. free of any compound which facilitates its introduction into cells (nucleic acid sequence transfer). However, in order to promote its introduction into target cells so as to obtain the genetically modified cells of the invention, this nucleic acid sequence can be in the form of a vector, and more particularly in the form of a viral vector such as, for example, an adenoviral or retroviral vector, or a vector derived from a poxvirus, in particular derived from the vaccinia virus or from the Modified Virus Ankara (MVA), or from a nonviral vector such as, for example, a vector consisting of at least one said nucleic acid sequence complexed with or conjugated to at least one carrier molecule or substance selected from the group consisting of a cationic amphiphile, in particular a cationic lipid, a cationic or neutral polymer, a protic polar compound in particular chosen from propylene glycol, polyethylene glycol, glycerol, ethanol and 1-methyl-L-2-pyrrolidone or their derivatives, and an aprotic polar compound in particular chosen from dimethyl sulfoxide (DMSO), diethyl sulfoxide, di-n-propyl sulfoxide, dimethylsulfone, sulfolane, dimethylformamide, dimethylacetamide, tetramethylurea and acetonitrile, or their derivatives.

Moreover, such vectors can also comprise screening elements which can make it possible to direct the transfer of nucleic acid sequence toward certain cell types or certain specific tissues (tumor cells, cells of the lung epithelium, hematopoietic cell, muscle cell, nerve cell, etc.). They can also make it possible to direct the transfer of an active substance toward certain preferred intracellular compartments such as the nucleus or mitochondria, for example. They can also be elements which facilitate penetration inside the cell or endosome lysis. Such screening elements are widely described in the literature. They can, for example, be all or part of lectins, of peptides, in particular the JTS-1 peptide (see patent application WO 94/40958), of oligonucleotides, of lipids, of hormones, of vitamins, of antigens, of antibodies, of ligands specific for membrane receptors, of ligands capable of reacting with an antiligand, of fusogenic peptides, of nuclear localization peptides, or of a combination of such compounds. In particular, they can be galactosyl residues which make it possible to target the asialoglycoprotein receptor at the surface of hepatic cells, ligands which can interact with receptors such as receptors for growth factors, receptors for cytokines, lectins, adhesion proteins, it can also be an antibody fragment such as the Fab fragment, an INF-7 fusogenic peptide derived from the Ha-2 subunit of influenza virus hemagglutinin (Plank et al., 1994, J. Biol. Chem. 269, 12918-12924), a nuclear localization signal derived from the SV40 virus T antigen or from the EBNA-1 protein of the Epstein Barr virus.

According to the invention, the antibody expressed at the surface of the target cells is capable of binding to a polypeptide which is present at the surface of a cytotoxic effector cell or of a helper T lymphocyte, in particular a CD4 helper T lymphocyte, and which is involved in the process of activation of such a cell, and more particularly to a receptor which is directly involved in such a process. As is described above, this phenomenon of activation of cytotoxic effector cells or of helper T lymphocytes is a determining element of the cell-mediated immune reaction. However, it should be noted that, in the context of the implementation of the present invention, it is not essential that the process of activation takes place after the binding by the antibody which is expressed according to the invention at the surface of the target cells. Specifically, in accordance with the invention, this antibody can also bind to peptides which are as defined, but which are present on already activated cytotoxic effector cells or helper lymphocytes. The expression “cytotoxic effector cells” is intended to refer to macrophages, cytotoxic T lymphocytes (TCLs) and killer cells (NKs), and to their derived cells such as, for example, LAKs (²Versteeg, 1992, Immunology Today, 13, 244-247; Brittende et al., 1996, Cancer 77, 1226-1243; Poplack et al., 1976, Blood 48, 809-816). The expression “helper T lymphocytes” is intended to refer to in particular CD4s which allow, after activation, the secretion of factors for activation of the effector cells of the immune response (see more above). The polypeptides, and in particular the receptors, which are expressed at the surface of these cells, and which are involved in the activation of such cells, consist in particular of all or part of the TCR complex, more particularly TCR-α, TCR-β or CD3, all or part of the CD8, CD4, CD28, LFA-1,4-1BB (Melero et al., 1998, Eur. J. Immunol., 28, 1116-1121), CD47, CD2, CD1, CD9, CD45, CD30 or CD40 complexes, all or part of the receptors for cytokines (Finke et al., 1998, Gene Therapy, 5, 31-39), such as IL-7, IL-4, IL-2, IL-15 or GM-CSF, all or part of the NK cell receptor complex, such as for example Vα14NKT (Kawano et al., 1998, Immunology, 95, 5690-5693), NKAR, Nkp46 (Pessino et al., 1998, J. Exp. Med., 188, 953-960) or Nkp44, all or part of macrophage receptors such as, for example, the Fc receptor (Deo et al., 1997, Immunology Today, 18, 127-135). According to one particular embodiment, it is also possible to envisage genetically modifying, in particular in vivo, cytotoxic effector cells or helper T lymphocytes so that they express at their surface a polypeptide which is not naturally expressed by these cells, and which is capable of inducing the process of activation of such cells, by introducing into these cells nucleic acid sequences containing the gene encoding such a polypeptide. In accordance with the present invention, it is then possible to select a nucleic acid sequence containing a gene of therapeutic interest encoding all or part of an antibody which can be expressed at the surface of the target cells of the patient to be treated, said antibody being capable of binding to such a polypeptide which is not naturally expressed by these cytotoxic effector cells or helper T lymphocytes.

More particularly, the present invention is based on the possibility of cloning the genes encoding all or part of an antibody and of expressing said antibody in cells after transfer of said genes into said cells using vectors as described above. The literature provides a large number of examples of genes encoding antibodies capable of reacting with such polypeptides or receptors. It is within the scope of persons skilled in the art to obtain the nucleic acid sequences encoding such antibodies. Mention is made, for example, of the genes encoding the light and heavy chains of the YTH 12.5 antibody (anti-CD3) (Routledge et al., 1991, Eur. J. Immuno. 21, 2717-2725) and of the anti-CD3 according to Arakawa et al., 1996, J. Biochem. 120, 657-662). The nucleic acid sequences of such antibodies are easily identifiable from the databases commonly used by persons skilled in the art.

It is also possible, using hybridomas which are available from ATCC and which secrete antibodies specific for polypeptides which are present at the surface of cytotoxic effector cells or helper T lymphocytes, and which are involved in the process of activation of such cells (for example a hybridoma excreting Gγ2b+κ immunoglobulins directed against TCR receptors), to clone the nucleic acid sequences encoding the heavy and/or light chains of these various antibodies by amplification methods such as RT-PCR using specific oligonucleotides, or techniques using cDNA libraries (Maniatis et al., 1982, Molecular cloning. A laboratory manual. C.S.H. Laboratory, Cold Spring Harbor, N.Y.). The sequences thus cloned are then available for their cloning into vectors. According to one preferred case of the invention, the nucleic acid sequence encoding the heavy chain of the antibody is fused by homologous recombination with the nucleic acid sequence encoding a transmembrane polypeptide, such as the rabies glycoprotein (these molecular biology techniques are fully described in French patent application No. FR 97 09152) or gp160 (Polydefkis et al., 1990, J. Exp. Med., 171, 875-887).

Among the mammalian target cells that the invention proposes to eliminate or whose progression it proposes to limit, mention may be made more specifically of tumor cells, cells infected with a viral pathogenic agent and cells infected with a bacterial pathogenic agent. According to the invention, the expression at the surface of these cells of all or part of an antibody capable of binding to a polypeptide which is present at the surface of a cytotoxic effector cell or of a helper T lymphocyte, and which is involved in the process of activation of such a cell, makes it possible to direct the cytotoxic immune response toward a given target, and more particularly to direct this response to a tumor or to an infectious site.

By way of “viral pathogenic agent”, mention may be made, for example, of the HIV, EBV and CMV virus, the hepatitis virus and papillomasviruses. By “parasitic pathogenic agent”, mention may be made, for example, of Leishmania lesmaniae and Plasmodium falciparum.

According to one specific embodiment, the invention relates to a biological material consisting of at least one target cell, such as in particular a tumor cell or a cell infected with a viral pathogenic agent, which does not naturally produce antibodies, in a form which allows their administration to the body of a human or animal mammal, and optionally their culturing beforehand, said cell being genetically modified in vitro with at least one nucleic acid sequence containing at least one gene encoding all or part of an antibody which is expressed at the surface of said target cell, and said antibody being capable of binding to a polypeptide which is present at the surface of a cytotoxic effector cell or of a helper T lymphocyte, such as those described above. More particularly, said target cell originates either from the mammal to be treated, or from a mammal other than the one to be treated. In the latter case, it should be noted that said target cell will have undergone a treatment making it compatible with the mammal to be treated. According to one preferred case, the term “mammal” is intended to refer to a human mammal.

Such a biological material, when it is administered to a patient, and more particularly administered via the intratumor route, is capable of inducing in this patient a cell-mediated immune response which can lead to the production of cytokines and to the cytotoxic effect of the effector cells, which cause not only the elimination of the administered cells but also the elimination of the neighboring cells presenting the antigens, in particular the tumor antigens, which can be recognized by said activated cytotoxic effector cells.

The invention relates, moreover, to the use of a biological material as described above for preparing a pharmaceutical composition intended for treating or for preventing cancers or viral infections. More particularly, the invention relates to the use of one nucleic acid sequence containing at least one gene of therapeutic interest and elements which ensure the expression of said gene in vivo in target cells genetically modified with a said nucleic acid sequence, said gene of therapeutic interest encoding all or part of an antibody which is expressed at the surface of said target cell, and which is capable of binding to a polypeptide which is present at the surface of a cytotoxic effector cell or of a helper T lymphocyte and which is involved in the process of activation of such a cell, for preparing pharmaceutical compositions intended for treating a mammal by gene transfer.

For implementing the treatment of the mammal mentioned in the present invention, it is possible to have pharmaceutical compositions comprising a biological material as described above which is advantageously combined with a pharmaceutically acceptable vehicle for administration to humans or to animals. The use of such supports is described in the literature. This pharmaceutically acceptable vehicle is preferably isotonic, hypotonic or has a weak hypertonicity and a relatively low ionic strength, such as for example a solution of sucrose. Moreover, said composition can contain solvents, aqueous or partially aqueous vehicles, such as sterile water which is free of pyrogenic agents, and dispersion media, for example. The pH of these pharmaceutical compositions is suitably adjusted and buffered according to conventional techniques.

According to one variant, the invention relates to a pharmaceutical composition comprising in particular a biological material as described and a protein compound which is naturally responsible for or involved in the activation of cytotoxic effector cells or of helper T lymphocytes. More particularly, such a compound will consist of a cytokine (The cytokine Handbook, 2nd Ed., Ed. A. W. Thomson, Ac. Press, Harcourt Brace & Company), a chemokine (Rollins et al., 1997, Blood, 90, 909-928; Devalaraja et al., 1999, TIPS, 20, 151-156) or any other compound which allows the costimulation of cytotoxic effector cells (for example, all or part of an anti-CD28 antibody; Stefan et al., 1997, Cancer research, 59(8), 1961-1967). Preferably, said compound will be IL2. In one implementation variant, the biological material according to the present invention may also comprise a DNA sequence which ensures the expression of a compound which is involved in the activation of cytotoxic effector cells or of helper T lymphocytes. In this case, this sequence can be contained in the nucleic acid sequence described above, or contained in a nucleic acid sequence which is independent of the one containing said gene of therapeutic interest (WO 95/09241).

According to a first possibility, the medicinal product can be administered directly in vivo, or via an ex vivo approach which consists in removing target cells from the mammal to be treated, in transfecting them in vitro according to the invention, and in readministering them to said mammal.

The biological material according to the invention can be administered in vivo in particular in injectable form, especially via the intratumor route. It is also possible to envisage an intratracheal, intranasal, epidermal, intravenous, intra-arterial, intramuscular, intrapleural or intracerebral injection using a syringe or any other equivalent means. According to another embodiment, it is possible to use systems which are suitable for treating the airways or mucous membranes, such as inhalation, instillation, or aerosolization via the topical route, via oral administration or any other means which is completely known to persons skilled in the art and which is applicable to the present invention. The administration can take place as a single dose or as repeated doses, one or more times after a certain period of delay. The most suitable route of administration and dose vary as a function of various parameters, such as for example the individual or the disease to be treated, or even of the nucleic acid to be transferred or of the target organ/tissues.

The invention also relates to a mammalian cell which does not naturally produce antibodies, characterized in that it is genetically modified with at least one nucleic acid sequence containing at least one gene of therapeutic interest and elements which ensure the expression of said gene in said cell, said gene of therapeutic interest encoding all or part of an antibody which is expressed at the surface of said genetically modified cell, and in that said antibody is capable of binding to a polypeptide which is present at the surface of a cytotoxic effector cell or of a helper T lymphocyte, and which is involved in the process of activation of such a cell described above.

Finally, the invention relates to a method for preparing a cell as described above, characterized in that at least one nucleic acid sequence containing at least one gene of therapeutic interest and elements which ensure the expression of said gene in said cell, said gene of therapeutic interest encoding all or part of an antibody which is expressed at the surface of said genetically modified cell, is introduced into a mammalian cell which does not naturally produce antibodies, by any suitable means, and in that said antibody is capable of binding to a polypeptide which is present at the surface of a cytotoxic effector cell or of a helper T lymphocyte, and which is involved in the process of activation of such a cell, and then in that, from these cells, those which are genetically modified with said nucleic acid sequence are chosen.

The examples below illustrate the invention without limiting it in any way.

Legend of the figures:

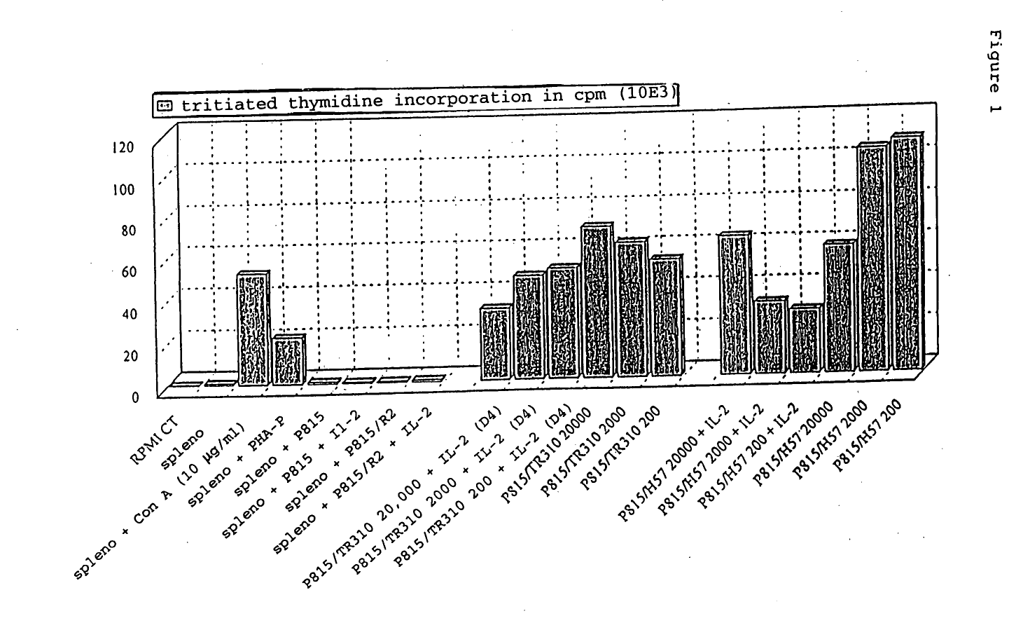

FIG. 1: Proliferation assay on murine splenocytes (2E5 cells/well) activated with P815 cells armed with the antibodies TR310 and H57-597. Presence together for 5 days. 8 h tritiated thymidine labeling. The results are expressed as amount of tritiated thymidine incorporated (10E3 cpm).

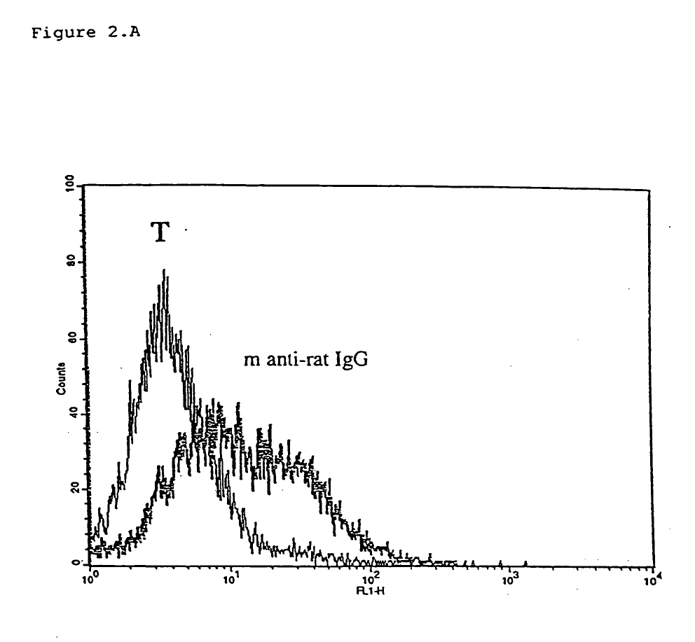

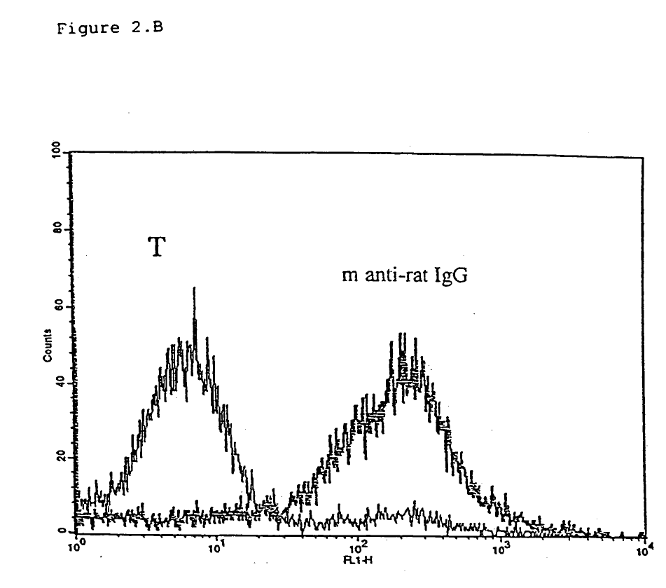

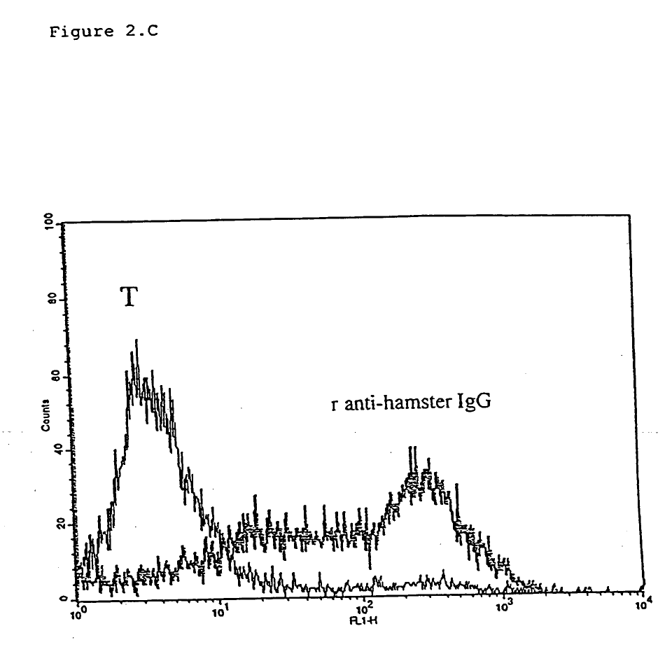

FIG. 2: Analysis by flow cytometry of the expression of the various antibodies after infection of BHK21 cells with the various recombinant viruses. A/B/C correspond to infections carried out with the MVATG14205, MVATG14240 and MVATG14237 viruses, respectively. (T: negative labeling using a negative control antibody; m anti-rat IgG: labeling using a mouse antibody directed against rat IgGs; r anti-hamster IgG: labeling using a rat antibody directed against hamster IgGs).

FIG. 2A: Analysis of the expression of the antibody TR310.

FIG. 2B: Analysis of the expression of the antibody KT3.

FIG. 2C: Analysis of the expression of the antibody H57-597.

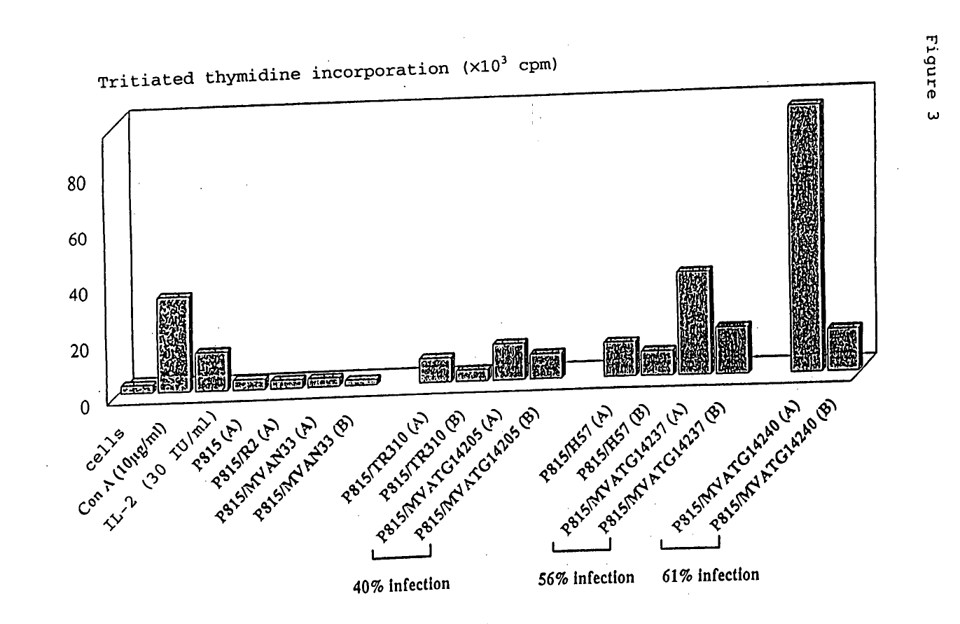

FIG. 3: P815 model (weak immunogenicity; H2d; MHC1; ICAM1; CD48). Proliferation assay on murine splenocytes (2×10⁵ cells/well) activated with P815 cells infected with the various recombinant MVA viruses. A/B corresponds to the bringing into contact of 2×10⁵ splenocytes with 20,000 or 2000 of the various P815 cells, respectively. P815/TR310, P815/H57-597 and P815/R2: P815s armed with the antibodies TR310, H57-597 and a control antibody, respectively; P815/MVATG14205, P815/MVATG14237, P815/MVATG14240: P815s infected with the various recombinant MVA viruses; Con A and IL-2: positive controls corresponding to the stimulation of splenocytes with concanavalin A or recombinant interleukin-2; cells: splenocytes only. The percentage of infection with the various recombinant MVA viruses is given.

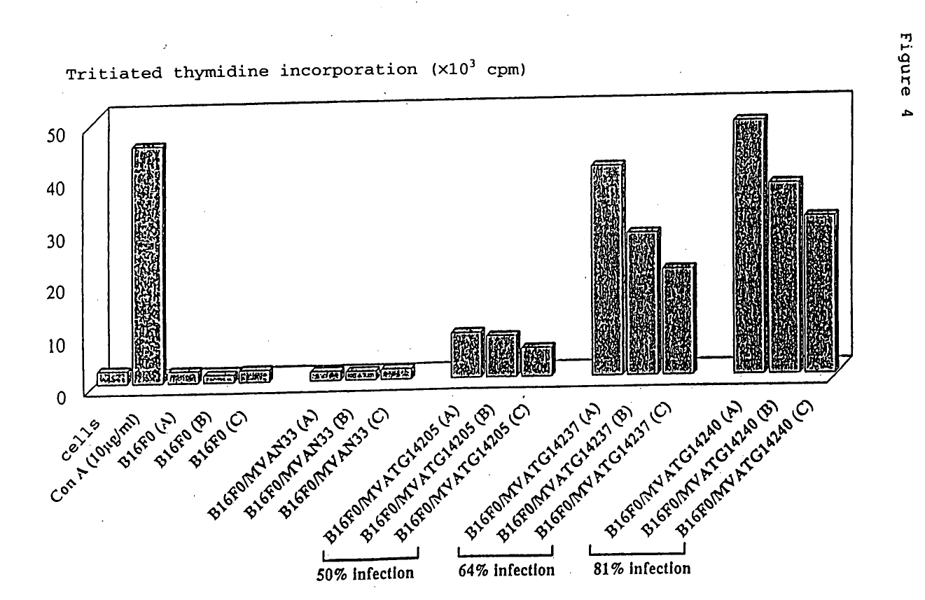

FIG. 4: B16F0 model (nonimmunogenic; H 2b) proliferation test on murine splenocytes (2×10⁵ cells/well) activated with B16F0 cells infected with the various recombinant MVA viruses. A/B/C corresponds to the bringing into contact of 2×10⁵ splenocytes with 20,000, 10,000 or 2000 of the various B16F0 cells. B16F0/MVATG14205, B16F0/MVATG14237, B16F0/MVATG14240: B16F0 infected with the various recombinant MVA viruses; Con A: positive control corresponding to the stimulation of splenocytes with concanavalin A; cells: splenocytes only. The percentage of infection with the various recombinant MVA viruses is given.

### EXAMPLE 1

1—Methods:

1-1 Construction of the Recombinant MVA Viruses

In order to allow the cloning of the nucleic acid sequences encoding various antibodies chosen for their capacity to activate T lymphocytes, three different hybridomas were selected:
hybridoma TR310 (rat anti-murine Vb7 (IgG2b); ATCC HB-219; I. L. Weissman; murine myeloma/rat splenocyte fusion).
hybridoma H57-597 (hamster anti-murine TCRab (IgG); ATCC HB-218; Kubo et al., 1989, J. Immunology, 142: 2736-2742; murine myeloma/hamster splenocyte fusion).
hybridoma KT3 (rat anti-murine CD3e (IgG2a); Tomonari et al., 1988, Immunogenetics, 28: 455-458; murine myeloma/rat splenocyte fusion).

The cloning of the sequences encoding all of the various heavy and light chains of these antibodies was carried out according to two different methods, using the total RNAs extracted from the 3 hybridomas:
a) by RT-PCR using specific oligonucleotides (Bca BEST™ RNA PCRKit, Takara Shuzo Co., Ltd; Frohman et al., 1988, Proc. Natl. Acad. Sci. USA, 85: 8998-9002) defined such that they hybridize at the conserved regions of the 3′ constant and 5′ variable sequences encoding the corresponding immunoglobulins (see C. A. Kettleborough et al., 1993, Eur. J. Immunol. 23: 206-211). More particularly, these sequences were defined with the aid of the following information available from GeneBank:

Hybridoma TR310 and KT3 (rat heavy and light chains):
gamma-type heavy chain: Rat anti-acetylcholine receptor antibody gene, rearranged Ig gamma-2a chain, VDJC region, complete cds. Author: Agius, M. A. and Bharati, S. 1993. Genebank access number=L22654
kappa-type light chain: Rat anti-acetylcholine receptor antibody gene, kappa-chain, VJC region, complete cds. Author: Agius, M. A. and Bharati, S. 1993. Unpublished. Genebank access number=L22653.

Hybridoma H57-597: (hamster heavy and light chain):
gamma-type heavy chain: Cricetulus migratorius IgG heavy chain mRNA, complete cds. Author: Whitters, M. J. and Collins, M. 1995. Immunogenetics. 42(3): 227-228. Genebank access number=U17166.
lambda-type light chain: Mus Musculus immunoglobulin lambda chain (IgL) mRNA, complete cds. Author: Reidl, L. S. Kinoshita, C. M. and Steiner, L. A. 1992. J. Immunol. 149: 471-480. Genebank access number=M94349.
b) by constructing cDNA libraries in pBluescript (Universal Riboclone System, Promega, Madison, USA; Maniatis et al., 1982, Molecular cloning. A laboratory manual. C.S.H. Laboratory, Cold Spring Harbor, N.Y.) and screening these libraries with the fragments amplified according to a).

The chains thus isolated are subcloned by recombination in a recombinant MVA virus containing the nucleic acid sequence encoding the transmembrane region of the rabies virus (Modified Vaccinia Ankara; Antoine et al., 1998, Virology, 244: 365-396 and French patent application No. FR 97 09152) in order to obtain recombinant viruses capable of expressing functional antibodies which are capable of recognizing and of binding to their specific antigen, and which are expressed in a transmembrane manner at the surface of the recombined cells.

1-2 In Vitro Study of the Effect of the Antibodies TR310 and H57-597 on the Proliferation of Murine Splenocytes.

Murine splenocytes (strain DBA/2) were stimulated with P815HT murine tumor cells (strain C57/B16; Acres et al., 1993, J. Immunother. 14: 136-143) in the presence of one or other of the two antibodies (TR310 and H57-597) purified from the corresponding hybridoma supernatants, for 1 h at 37° C. The presence at the surface of the P815HT cells of immunoglobulin receptors of Fc type enables said cells to be covered with the antibodies. After 5 days of incubation between the P815 cells presenting the selected antibodies at their surface and the splenocytes, the proliferation of the T lymphocytes is measured using a tritiated thymidine incorporation assay (Gimmi et al., 1996, Nat. Med. 12: 1367-1371).

Moreover, in this study, several parameters were analyzed:
a) number of P815 cells presenting at their surface the selected antibodies required for the stimulation;
b) possible costimulation with the aid of human recombinant IL-2 on day 4 (100 ng/ml i.e. 50 IU/ml; R&Dsystems).
   1-3 Study of the Th (T Helper) Response Associated with the Stimulation of the Splenocytes by the Various Antibodies (TR310 and H57-597) Presented at the Surface of the P815 Cells.

In order to evaluate this response, the supernatants from the proliferation assay above, carried out in the presence of human recombinant IL-2, were sampled on day 4 or 5. The presence of IL-4 (Th2-type response) or of IFNγ (Th1-type response) in these supernatants was measured using commercially available specific kits, according to the manufacturer's recommendations (Quantikine Kit, R&D systems).

2—Results

2-1 In Vitro Study of the Effect of the Antibodies TR310 and H57-597 on the Proliferation of Murine Splenocytes.

2-1-1—Controls

In parallel with this study, a certain number of controls were also analyzed.

It involves more particularly measuring the effects observed under conditions which are identical to the assay itself of:

RPMI CT: negative control containing only the medium.

Spleno: negative control containing only the splenocyte cells.

Spleno+P815+/−IL-2: negative control containing the splenocyte cells+the unarmed P815 target cells, in the presence or absence (+/−) of IL-2 (100 ng/ml) added on day 4.

Spleno+P815/R2+/−IL-2: negative control containing the splenocyte cells+the P815 target cells bearing at their surface a neutral antibody (not capable of inducing lymphocyte activation)+/−IL-2 (100 ng/ml) added on day 4.

Spleno+ConA: positive control corresponding to the stimulation of the murine splenocytes using Concanavalin A (10 μg/ml), which is a powerful murine mitogen.

Spleno+PHA-P: positive control corresponding to the stimulation of the murine splenocytes using phytohemagglutinin A (1 μg/ml), which is a powerful human mitogen.

2-1-2—Assay

P815/TR310 (20,000/2000/200)+/−IL-2: Evaluation of the stimulation of murine splenocytes by P815 target cells bearing at their surface a TR310 antibody (2E4, 2E3, 2E2 cells/assay), in the presence or absence of human recombinant IL-2 from D+4 onward.

P815/H57-597 (20,000/2000/200)+/−IL-2: Evaluation of the stimulation of murine splenocytes by P815 target cells bearing at their surface a H57-597 antibody (2E4, 2E3, 2E2 cells/assay), in the presence or absence of human recombinant IL-2 from D+4 onward.

The stimulation results obtained are presented in FIG. 1. These results show that the presentation of “activating” antibodies for T lymphocytes at the surface of P815 target cells allows the activation and proliferation of murine splenocytes (visualized by the incorporation of tritiated thymidine). The addition of human recombinant interleukin-2 does not appear to amplify this antibody-dependent stimulation. Moreover, we have also shown that the lymphocyte activation as measured is accompanied by lysis of the P815 target cells.

2-2 Study of the Th (T Helper) Response Associated with the Stimulation of the Splenocytes by the Various Antibodies (TR310 and H57-597).

The results observed are presented in the following Table 1 which gives the measurements of the IL-4 and IFNγ doses after activation of murine splenocytes by the antibodies TR310 and H57-597 present at the surface of P815 murine tumor cells.

| | P815 | P815-TR310 | P815-H57-597 |
|---|---|---|---|
| Activating | 2000 | 2000 | 2000 |
| cells | cells/assay | cells/assay | cells/assay |
| IL-4 dose | 0 | 31 | 23.5 |
| produced in | | | |
| pg/ml | | | |
| IFNg dose | 0 | 92.9 | 162.75 |
| produced in | | | |
| pg/ml | | | |

These doses show that the antibodies TR310 and H57-597, in a membrane presentation context, induce the 2 types of response Th1 and Th2 (Table 1). The antibodies TR310 and H57-597 are thus capable, when they are presented at the surface of tumor cells, of activating a Th1, but also a Th2, “helper”-type response by binding to their membrane receptor present at the surface of the CD4+ T cells. The secretion of these cytokines makes it possible in particular to increase the immune response directed against the tumor cells.

### EXAMPLE 2

1—Methods:

1-1 Construction of the Recombinant MVA Viruses

Three recombinant MVA viruses were constructed (MVATG14205 expressing the rat antibody TR310 (anti-murine V beta 7); MVATG14237 expressing the hamster antibody H57-597 (anti-hamster TCR alpha/beta) and MVATG14240 expressing the rat KT3 antibody (anti-rat CD3 epsilon). The expression cassettes were introduced into deletion II of the MVA (Modified Virus Ankara) as described in patent application WO 9903885. The light chain of each antibody was placed under the control of the early/late vaccinia promoter p7.5 (Goebel et al., 1990, Virology, 179, 247-266). The sequence encoding the heavy chain of each antibody was placed under the control of the early/late vaccinia promoter pH5R (Goebel et al., 1990, Virology, 179, 247-266). In order to facilitate the selection of the recombinant MVA viruses, the GPT (xanthine-guanine phosphoribosyl-transferase) selection gene of E. coli is used. The C-terminal end of the heavy chain of each antibody is also fused with the transmembrane and intracytoplasmic domain of the rabies glycoprotein in order to allow the anchoring of the antibodies in the plasma membrane.

1-2′ Evaluation of the Expression of the Various Antibodies After Infection of Chicken Embryonic Cells (CECs) with the Various Recombinant MVA Viruses.

The expression is analyzed by Western Blot, respecting the conditions of the “ECL™ Western Blotting” kit from Amersham Life Science (UK). For this, chicken embryonic cells are infected, at a multiplicity of infection (MOI) of 1, with the various recombinant MVA viruses. The same cells are also infected with the control virus MVAN33. After infection for 24 h, the cells are washed with PBS, and then subjected to sonication in the loading buffer. The suspension is then denatured for 3 min at 95° C., before being fractionated on a 13% polyacrylamide gel. The proteins thus fractionated are then transferred onto a PVDF membrane (porablot; Macherey-Nagel). The expression of the antibodies TR310 and KT3 is analyzed after hybridization using an HRPO-coupled mouse antibody (horseradish peroxidase; 10 μg/ml) directed against rat IgGs. The expression of the antibody H57-597 is analyzed after hybridization using an HRPO-coupled rat antibody directed against hamster IgGs.

1-3 Analysis by Flow Cytometry of the Expression of the Various Antibodies After Infection of BHK21 Cells with the Various Recombinant Viruses.

The expression is analyzed by FACS, after infection of BHK21 cells (Baby Hamster Kidney), at an MOI of 1, with the various recombinant MVA viruses. After infection for 12 h, the cells are detached and then analyzed. Detection of the antibodies TR310 and KT3 is carried out after incubation with an FITC-coupled mouse antibody directed against rat IgGs (Jackson ImmunoResearch Laboratories Inc (Pennsylvania; USA)). Detection of the antibody H57-597 is carried out using an FITC-coupled rat antibody directed against hamster IgGs (Jackson ImmunoResearch Laboratories Inc (Pennsylvania; USA)). The infected cells were also incubated with a control antibody.

1-4 In Vitro Study of the Functionality of the Various Recombinant MVA Viruses.

The functionality of the recombinant MVA viruses is tested by carrying out proliferation assays on murine splenocytes. Murine splenocytes (strain DBA/2) are stimulated with P815HT or B16F0 murine tumor cells infected with the 3 recombinant MVA viruses. For this, P815HT and B16F0 cells are infected, at an MOI of 1, for 20 h with the MVATG14205, MVATG14237, MVATG14240 and MVAN33 (negative control) viruses. For the P815HT model, the cells are also brought into contact with 10 μg/ml of the antibodies TR310 and H57-597 purified from hybridomous supernatant. The infected or armed cells are then treated for 1 h with mitomycin C (Sigma; 50 μg/ml) in order to stop their division. After incubation for 5 days under the various conditions of P815 and B16F0 cells and the murine splenocytes, the proliferation of the T lymphocytes is measured using a tritiated thymidine incorporation assay.

2—Results:

2-1 Construction of the Recombinant MVAs

The recombinant MVA viruses (MVATG14205; MVATG14237 and MVATG14240) are generated by homologous recombination in chicken embryonic cells with the aid of the various transfer plasmids (pTG14205; pTG14237 and pTg14240) and of a wild-type MVA virus MVAN33 as described in patent application WO 9903885.

2-2 Evaluation of the Expression of the Various Antibodies after Infection of Chicken Embryonic Cells (CECs) with the Various Recombinant MVA Viruses.

The expression of the various antibodies (TR310, H57-597 and KT3) is analyzed by Western Blot after infection of CECs with the recombinant MVA viruses. The results observed show that there is indeed rat IgG-type immunoglobulin expression in the cells infected with the MVATG14-205 and MVATG14240 viruses. After infection with MVATG14237, there is also expression of a hamster IgG-type immunoglobulin. These results are revealed by the presence of a specific band which migrates at approximately 60 kDa, and which corresponds to the heavy chain of the immunoglobulin. No specific band could be observed after infection of CECs with the control virus MVAN33.

2-3 Analysis by Flow Cytometry of the Expression of the Various Antibodies After Infection of BHK21 Cells with the Various Recombinant Viruses.

The expression and assembly of the 3 antibodies are evaluated by flow cytometry (FIG. 2). BHK21 cells are infected, at an MOI of 1, with the 3 recombinant viruses. The FACS analysis made it possible to demonstrate the expression of surface IgG of hamster (H57-597) and of rat (TR310 and KT3) after incubation of the infected cells with antibodies directed specifically against rat and hamster IgGs. This analysis also made it possible to show the expression of kappa-type light chain in the BHK21 cells infected with the MVATG14205 and MVATG14240 viruses. These results make it possible to show that the infection of BHK21 cells by the various recombinant MVA viruses leads to the expression of correctly rearranged membrane immunoglobulins.

2-4 In Vitro Study of the Functionality of the Various Recombinant MVA Viruses.

The functionality of the recombinant MVA viruses is tested by carrying out proliferation assays on murine splenocytes. For this, 2 types of murine model are used; P815HT cells (FIG. 3) and B16F0 cells (FIG. 4). These 2 types of cell are infected with the recombinant viruses in order to express, at their surface, transmembrane antibodies directed against all or part of the murine TCR/CD3 complex. We measured the proliferation induced by bringing infected P815HT and B16F0 cells into contact with naive murine splenocytes with a similar haplotype. First of all, P815HT cells (weakly immunogenic; H2d; expressing MHCI, ICAMI and CD48) are infected with the 3 recombinant viruses and a control virus MVAN33. In parallel, the same P815HT cells are incubated with the antibodies TR310 and H57-597 in order to arm them with antibodies via their Fc receptor. For the MVATG14205 virus (stimulation of murine Vbeta 7 cells), the stimulation induced by infected P815 cells is slightly greater than that obtained with the armed cells. In addition, the level of proliferation remains very low, as expected with the specificity of the antibody. With the MVATG14237 and MVATG14240 viruses, the index obtained with the infected cells is clearly higher than that obtained with the armed cells. The indices obtained with the cells infected with the 3 viruses are very clearly higher than those obtained with powerful mitogenic agents such as concanavalin A or interleukin-2. Finally, we were able to demonstrate, in all cases, a dose-response proportional to the number of infected cells brought into contact with the splenocytes. No stimulation could be observed with P815HT cells which were noninfected, armed with a control antibody or infected with a control virus.

In an identical manner, B16F0 cells are used in the same type of experiment (FIG. 4). These B16F0 murine cells are nonimmunogenic and have no costimulation molecule. We thus demonstrated a strong proliferation of naive splenocytes after contact with infected cells. In an identical manner, the cells infected with MVATG14205 induce less proliferation than the cells infected with MVATG14237 and MVATG14240 viruses. For the latter two, the proliferation index obtained with 20,000 cells is comparable to that obtained with Con A. In this model, the amount of infected cells also appears to have an effect on the proliferation index obtained (dose-response).

In conclusion, it appears very clearly that the 3 recombinant viruses are functional. The expression of the antibodies TR310, H57-597 and KT3 at the surface of cells makes it possible to induce, according to the invention, a strong proliferation of naive T cells. This stimulation is advantageously greater than that obtained with cells armed with the same antibodies via the Fc-type receptor, but also than that obtained with powerful mitogenic agents such as Con A.

1. Biological material for preparing pharmaceutical compositions intended for treating mammals, comprising:
either at least one nucleic acid sequence containing at least one gene of therapeutic interest and elements which ensure the expression of said gene in vivo in target cells intended to be genetically modified with said nucleic acid sequence;
or at least one target cell which does not naturally produce antibodies and which is genetically modified in vitro with at least one nucleic acid sequence above,
characterized in that said gene of therapeutic interest encodes all or part of an antibody which will be expressed at the surface of said target cell, and in that said antibody is capable of binding to a polypeptide which is present at the surface of a cytotoxic effector cell or of a helper T lymphocyte, and which is involved in the process of activation of such a cell.

2. Biological material according to claim 1, characterized in that said nucleic acid sequence is in the form of a naked DNA or RNA sequence.

3. Biological material according to claim 1, characterized in that said nucleic acid sequence is a vector which allows the transfer of said gene of therapeutic interest into said target cells.

4. Biological material according to claim 3, characterized in that said vector is a viral vector.

5. Biological material according to claim 4, characterized in that said viral vector is an adenoviral or retroviral vector, or a poxvirus, in particular derived from the vaccinia virus or from the Modified Virus Ankara (MVA).

6. Biological material according to claim 3, characterized in that said vector consists of at least one said nucleic acid sequence complexed with or conjugated to at least one carrier molecule or substance selected from the group consisting of a cationic amphiphile, in particular a cationic lipid, a cationic or neutral polymer, a protic polar compound in particular chosen from propylene glycol, polyethylene glycol, glycerol, ethanol and 1-methyl-L-2-pyrrolidone or their derivatives, and an aprotic polar compound in particular chosen from dimethyl sulfoxide (DMSO), diethyl sulfoxide, di-n-propyl sulfoxide, dimethylsulfone, sulfolane, dimethylformamide, dimethylacetamide, tetramethylurea and acetonitrile, or their derivatives.

7. Biological material according to any one of the preceding claims, characterized in that said nucleic acid sequence contains a gene encoding the heavy chain of an antibody capable of binding to a polypeptide which is present at the surface of a cytotoxic effector cell or of a helper T lymphocyte, and which is involved in the process of activation of such a cell, fused with a transmembrane polypeptide.

8. Biological material according to claim 7, characterized in that said nucleic acid sequence also contains a gene encoding the light chain of an antibody capable of binding to a polypeptide which is present at the surface of a cytotoxic effector cell or of a helper T lymphocyte, and which is involved in the process of activation of such a cell.

9. Biological material according to either of claims 7 and 8, characterized in that said transmembrane polypeptide is selected from the group consisting of a glycoprotein, a lipoprotein and a membrane receptor.

10. Biological material according to claim 9, characterized in that said transmembrane polypeptide is selected from the group consisting of the rabies virus glycoprotein, gp160 and CD4.

11. Biological material according to any one of the preceding claims, characterized in that said polypeptide which is present at the surface of a cytotoxic effector cell or of a helper T lymphocyte, and which is involved in the process of activation of such a cell, is a receptor.

12. Biological material according to claim 11, characterized in that said cytotoxic effector cell is selected from the group consisting of macrophages, cytotoxic T lymphocytes (TCLs) and killer cells (NKs), or their derived cells.

13. Biological material according to either of claims 11 and 12, characterized in that said receptor is selected from the group consisting of all or part of the TCR complex, more particularly TCR-α, TCR-β or CD3, CD8, CD4, CD28, LFA-1,4-1BB, CD47, CD2, CD9, CD45, CD40, receptors for cytokines, such as IL-7, IL-4, IL-2, IL-15 or GM-CSF, Vα14NKT, NKAR and the Fc receptor.

14. Biological material according to any one of the preceding claims, characterized in that said target cell is a mammalian tumor cell, a mammalian cell infected with a viral pathogenic agent, or a mammalian cell infected with a bacterial pathogenic agent.

15. Biological material according to claim 1, characterized in that it consists of at least one target cell which does not naturally produce antibodies, in a form which allows their administration to the body of a mammal, and optionally their culturing beforehand, said cell being genetically modified in vitro with at least one nucleic acid sequence containing at least one gene encoding all or part of an antibody which is expressed at the surface of said target cell, and in that said antibody is capable of binding to a polypeptide which is present at the surface of a cytotoxic effector cell or of a helper T lymphocyte, and which is involved in the process of activation of such a cell.

16. Biological material according to claim 15, characterized in that said target cells originate from the mammal to be treated.

17. Biological material according to claim 15, characterized in that said target cells originate from a mammal other than the one to be treated and have undergone a treatment making them compatible.

18. Biological material according to one of claims 1 to 17, characterized in that it also comprises at least one DNA sequence which ensures the expression of a compound which is involved in the activation of cytotoxic effector cells or of helper T lymphocytes.

19. Use of a biological material according to one of claims 1 to 18 for preparing a pharmaceutical composition intended for treating or for preventing cancers or viral infections.

20. Use of a nucleic acid sequence containing at least one gene of therapeutic interest and elements which ensure the expression of said gene in vivo in target cells genetically modified with a said nucleic acid sequence, said gene of therapeutic interest encoding all or part of an antibody which is expressed at the surface of said target cell, and which is capable of binding to a polypeptide which is present at the surface of a cytotoxic effector cell or of a helper T lymphocyte, and which is involved in the process of activation of such a cell, for preparing pharmaceutical compositions intended for treating a mammal by gene transfer.

21. Pharmaceutical composition comprising a biological material according to one of claims 1 to 18, advantageously in combination with a pharmaceutically acceptable vehicle.

22. Pharmaceutical composition according to claim 21, comprising a biological material according to one of claims 15 to 17 and a compound which is naturally responsible for the activation of cytotoxic effector cells or of helper T lymphocytes.

23. Pharmaceutical composition according to claim 22, characterized in that said compound is a cytokine or a chemokine.

24. Mammalian cell which does not naturally produce antibodies, characterized in that it is genetically modified with at least one nucleic acid sequence containing at least one gene of therapeutic interest and elements which ensure the expression of said gene in said cell, said gene of therapeutic interest encoding all or part of an antibody which is expressed at the surface of said genetically modified cell, and in that said antibody is capable of binding to a polypeptide which is present at the surface of a cytotoxic effector cell or of a helper T lymphocyte, and which is involved in the process of activation of such a cell.

25. Method for preparing a cell according to claim 24, characterized in that at least one nucleic acid sequence containing at least one gene of therapeutic interest and elements which ensure the expression of said gene in said cell, said gene of therapeutic interest encoding all or part of an antibody which is expressed at the surface of said genetically modified cell, is introduced into a mammalian cell which does not naturally produce antibodies, by any suitable means, and in that said antibody is capable of binding to a polypeptide which is present at the surface of a cytotoxic effector cell or of a helper T lymphocyte, and which is involved in the process of activation of such a cell, and then in that, from these cells, those which are genetically modified with said nucleic acid sequence are chosen.
